(19) United States
(12) Patent Application Publication
(10) Pub. No.: US 2026/0078312 A1
(43) Pub. Date: Mar. 19, 2026

(54) PROCESS FOR PRODUCING LIQUEFIED PETROLEUM GAS

(71) Applicant: UOP LLC, Rosemont, IL (US)

(72) Inventors: Neeraj Tiwari, Gurgaon (IN); Prakhar Agrawal, Gurgaon (IN)

(21) Appl. No.: 19/279,752

(22) Filed: Jul. 24, 2025

Related U.S. Application Data
(60) Provisional application No. 63/695,195, filed on Sep. 16, 2024.

(51) Int. Cl. C10L 1/04; C07C 7/09

(52) U.S. Cl. CPC C10L 1/04; C07C 7/09

(57) ABSTRACT

A process of producing liquefied petroleum gas is disclosed. The process comprises providing a stream comprising C4-hydrocarbons and at least 10 wppm hydrogen sulfide. The stream is fractionated in a fractionation column to provide an overhead stream comprising C2-hydrocarbons and a bottoms stream comprising C3-C4 hydrocarbons. A predominance of the hydrogen sulfide is separated and taken in the overhead stream. The bottoms stream can be taken as liquefied petroleum gas stream which comprises a permissible level of about 1 to about 30 wppm hydrogen sulfide.

### FIELD

The field is related to a process of producing liquefied petroleum gas. Particularly, the field may relate to a process of producing liquefied petroleum gas that meets a hydrogen sulfide specification.

### BACKGROUND

As the demand for fuel increases worldwide, there is increasing interest in producing fuels and blending components from sources other than crude oil. Often referred to as biorenewable sources, these sources include, but are not limited to, plant oils such as corn, rapeseed, canola, soybean, microbial oils such as algal oils, animal fats such as inedible tallow, fish oils and various waste streams such as yellow and brown greases and sewage sludge. A common feature of these sources is that they are composed of glycerides and free fatty acids (FFA). Both triglycerides and the FFAs contain aliphatic carbon chains having from about 8 to about 24 carbon atoms. The aliphatic carbon chains in triglycerides or FFAs can be fully saturated, or mono, di or poly-unsaturated.

Hydroprocessing can include processes which convert hydrocarbons in the presence of hydroprocessing catalyst and hydrogen to more valuable products. Hydrotreating is a process in which hydrogen is contacted with hydrocarbons in the presence of hydrotreating catalysts which are primarily active for the removal of heteroatoms, such as sulfur, nitrogen, oxygen and metals from the hydrocarbon feedstock. In hydrotreating, hydrocarbons with double and triple bonds such as olefins may be saturated.

The production of hydrocarbon products in the diesel boiling range can be achieved by hydrotreating a biorenewable feedstock. A biorenewable feedstock can be hydroprocessed by hydrotreating to deoxygenate, decarboxylate and/or decarbonylate the oxygenated hydrocarbons. Decarboxylation and decarbonylation remove a carbon from the paraffin molecule; whereas, deoxygenation does not. Hydrotreating may be followed by hydroisomerization to improve cold flow properties of diesel and jet fuel products. Hydroisomerization or hydrodewaxing is a hydroprocessing process that increases the alkyl branching on a hydrocarbon backbone in the presence of hydrogen and hydroisomerization catalyst to improve cold flow properties of the hydrocarbon. Hydroisomerization includes hydrodewaxing herein.

Hydrocracking is a hydroprocessing process in which hydrocarbons crack in the presence of hydrogen and hydrocracking catalyst to lower the molecular weight of hydrocarbons. Depending on the desired output, a hydrocracking unit may contain one or more beds of the same or different catalyst.

When producing jet fuel from triglycerides (also referred to as “fats”) a certain degree of hydrocracking and isomerization is needed to meet the specifications of jet fuel as outlined in ASTM D7566 Annex 2 and ASTM D1655. These key specifications that are required of the jet fuel in D7566 are freeze point of no higher than −40° C. (ASTM D5972, D7153 or D7154), density of no more than 772 kg/m³ (ASTM D1298 or D4052), T10 of less than 205° C. (ASTM D86), and a final boiling point (FBP) of less than 300° C. (ASTM D86). Larger molecules that do not meet these jet fuel specifications are hydrocracked primarily to meet these specifications which typically results in lower jet fuel yield and a low energy-density fuel which are both undesirable. Aviation fuel is valued for its high energy per volume.

Liquefied petroleum gas is colorless and odorless, and is widely used as fuel for households, businesses, industries, and automobiles. Liquefied petroleum gas primarily comprises propane (C₃H₈) and butane (C₄H₁₀). Liquefied petroleum gas can be separated from a fractionated off-gas stream downstream of a hydroprocessing unit. The recovered liquefied petroleum gas typically comprises hydrogen sulfide which needs to be separated to meet the fuel gas specification. An efficient and economical process is required for purifying liquefied petroleum gas before its use.

As refiners seek to add capability for processing biorenewable feedstocks, processes are sought to produce greater volumes of jet fuel due to its high value and demand. Processes for producing liquefied petroleum gas, diesel, and jet fuel from biorenewable feedstocks are desired.

### SUMMARY

We have found an alternate and improved process for removing hydrogen sulfide from liquefied petroleum gas (LPG) that does not require amine treating to produce LPG meeting the hydrogen sulfide specification. We have found that a deethanizer column can be used for liquefied petroleum gas treatment. The deethanizer column efficiently separates hydrogen sulfide from liquefied petroleum gas. The treated liquefied petroleum gas meets the hydrogen sulfide specification.

### BRIEF DESCRIPTION OF THE DRAWINGS

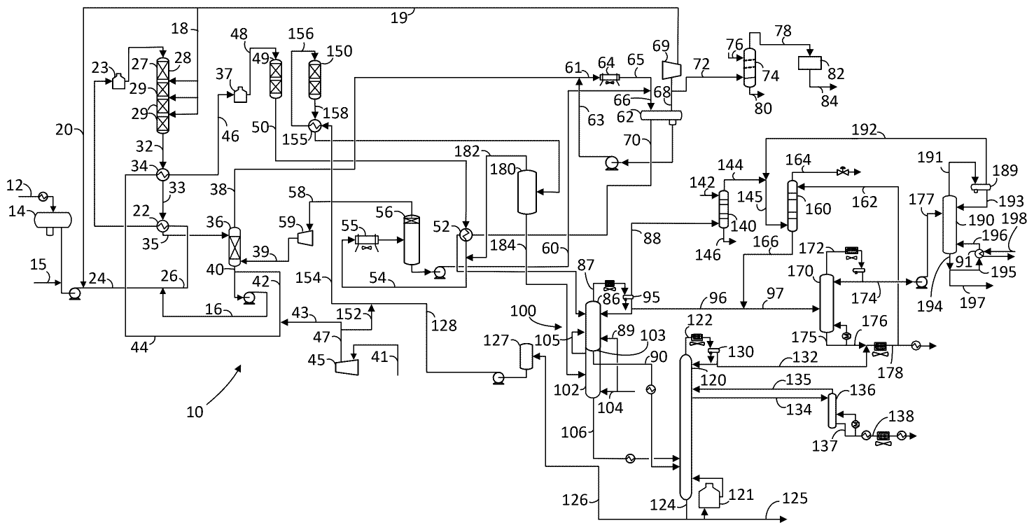

The FIG. 1 is a schematic process flow diagram of a process and apparatus of producing liquefied petroleum gas in accordance with the present disclosure.

### Definitions

The term “communication” means that material flow is operatively permitted between enumerated components.

The term “downstream communication” means that at least a portion of material flowing to the subject in downstream communication may operatively flow from the object with which it communicates.

The term “upstream communication” means that at least a portion of the material flowing from the subject in upstream communication may operatively flow to the object with which it communicates.

The term “direct communication” or “directly” means that flow from the upstream component enters the downstream component without passing through a fractionation or conversion unit to undergo a compositional change due to physical fractionation or chemical conversion.

The term “indirect communication” means that flow from the upstream component enters the downstream component after passing through a fractionation or conversion unit to undergo a compositional change due to physical fractionation or chemical conversion.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take a main product from the bottom.

As used herein, the term “a component-rich stream” means that the rich stream coming out of a vessel has a greater concentration of the component than the feed to the vessel.

As used herein, the term “a component-lean stream” means that the lean stream coming out of a vessel has a smaller concentration of the component than the feed to the vessel.

As used herein, the term “boiling point temperature” means atmospheric equivalent boiling point (AEBP) as calculated from the observed boiling temperature and the distillation pressure, as calculated using the equations furnished in ASTM D86 or ASTM D2887.

As used herein, the term “True Boiling Point” (TBP) means a test method for determining the boiling point of a material which corresponds to ASTM D-2892 for the production of a liquefied gas, distillate fractions, and residuum of standardized quality on which analytical data can be obtained, and the determination of yields of the above fractions by both mass and volume from which a graph of temperature versus mass % distilled is produced using fifteen theoretical plates in a column with a 5:1 reflux ratio.

As used herein, the term “T5” or “T90” means the temperature at which 5 mass percent or 90 mass percent, as the case may be, respectively, of the sample boils using ASTM D-86 or TBP.

As used herein, the term “initial boiling point” (IBP) means the temperature at which the sample begins to boil using ASTM D2887, ASTM D-86 or TBP, as the case may be.

As used herein, the term “final boiling point” (FBP) means the temperature at which the sample has all boiled off using ASTM D2887, ASTM D-86 or TBP, as the case may be.

As used herein, the term “jet fuel range material” means hydrocarbons boiling in the range of an IBP between about 85° C. (185° F.) and about 135° C. (275° F.) or a T5 between about 110° C. (230° F.) and about 160° C. (320° F.) and the “recycle cut point” comprising a T95 between about 295° C. (563° F.) and about 315° C. (599° F.) using the TBP distillation method. Hydrocarbons beyond the “recycle cut point” and up to the “diesel cut point” comprising a T95 between about 343° C. (650° F.) and about 399° C. (750° F.) are the “diesel boiling range” material using the TBP distillation method.

As used herein, the term “diesel boiling range” means hydrocarbons boiling in the range of an IBP between about 125° C. (257° F.) and about 175° C. (347° F.) or a T5 between about 150° C. (302° F.) and about 200° C. (392° F.) and the “diesel cut point” comprising a T95 between about 343° C. (650° F.) and about 399° C. (750° F.) using the TBP distillation method.

As used herein, the term “diesel conversion” means conversion of feed that boils above the diesel cut point to material that boils at or below the diesel cut point in the diesel boiling range.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

As used herein, the term “predominant” or “predominate” means greater than 50%, suitably greater than 75% and preferably greater than 90%.

As used herein, the term “Cₓ” are to be understood to refer to molecules having the number of carbon atoms represented by the subscript “x”. Similarly, the term “Cₓ−” refers to molecules that contain less than or equal to x and preferably x and less carbon atoms. The term “Cₓ+” refers to molecules with more than or equal to x and preferably x and more carbon atoms.

As used herein, the term “carbon number” refers to the number of carbon atoms per hydrocarbon molecule and typically a paraffin molecule.

### DETAILED DESCRIPTION

In the FIGURE, in accordance with an exemplary embodiment, a process 10 is shown for producing liquefied petroleum gas. A feed line 12 transports a hydrocarbon stream comprising a hydrocarbon feedstock into a feed surge drum 14. Preferably, the hydrocarbon feedstock is a biorenewable hydrocarbon feedstock. The biorenewable feedstock may be blended with a mineral feed stream but preferably comprises a predominance of biorenewable feedstock. A mineral feedstock is a conventional feed derived from crude oil that is extracted from the ground. The biorenewable feedstock may comprise a nitrogen concentration of about 50 wppm to about 2000 wppm. The biorenewable feedstock may comprise high oxygen content which can be up to 10 wt % or higher. The biorenewable feedstock may also comprise about 1 to about 500 wppm sulfur, typically no more than about 200 wppm sulfur.

A variety of different biorenewable feedstocks may be suitable for the process 10. The term “biorenewable feedstock” is meant to include feedstocks other than those obtained from crude oil. The biorenewable feedstock may include any of those feedstocks which comprise at least one of the glycerides and free fatty acids. Most of the glycerides will be triglycerides, but monoglycerides and diglycerides may be present and processed as well. Free fatty acids may be obtained from phospholipids which may source phosphorous in the feedstock. Examples of these biorenewable feedstocks include, but are not limited to, camelina oil, canola oil, corn oil, soy oil, rapeseed oil, soybean oil, colza oil, tall oil, sunflower oil, hempseed oil, olive oil, linseed oil, coconut oil, babassu oil, castor oil, peanut oil, palm oil, mustard oil, tallow, yellow and brown greases, lard, train oil, fats in milk, fish oil, algal oil, sewage sludge, and the like. Additional examples of biorenewable feedstocks include non-edible vegetable oils from the group comprising Jatropha curcas (Ratanjot, Wild Castor, Jangli Erandi), Madhuca indica (Mohuwa), Pongamia pinnata (Karanji, Honge), calophyllum inophyllum, moringa oleifera and Azadirachta indica (Neem). The triglycerides and FFAs of the typical vegetable or animal fat contain aliphatic hydrocarbon chains in their structure which have about 8 to about 30 carbon atoms. Biorenewable feedstocks may also include biomass pyrolysis oils and Fischer-Tropsch waxes. As will be appreciated, the biorenewable feedstock may comprise a mixture of one or more of the foregoing examples. The biorenewable feedstock may be pretreated to remove contaminants and filtered to remove solids.

The hydrocarbon stream in feed line 12 flows from the feed surge drum 14 via a charge pump perhaps after injection with a sulfiding agent in line 15 and mixes with a recycle hydrotreating hydrogen stream in a hydrotreating hydrogen line 20 to provide a combined hydrocarbon stream in line 24. The combined hydrocarbon stream in line 24 is mixed with a hydrotreating recycle stream in a recycle line 16 to provide a hydrotreating charge hydrocarbon stream in a hydrotreating charge line 26. The hydrotreating charge hydrocarbon stream in line 26 may be preheated in a combined feed exchanger 22 by heat exchange with a hydrotreated stream in line 33 and perhaps then in a fired heater 23. The heated hydrotreating charge hydrocarbon stream in the hydrotreating charge line 26 may be then charged to a hydrotreating reactor 28.

The hydrotreating reactor 28 may include a guard bed reactor or a guard bed 27. In FIG. 1, the hydrotreating reactor 28 includes a guard bed 27. The guard bed reaction temperature may range between about 246° C. (475° F.) and about 343° C. (650° F.) and suitably between about 288° C. (550° F.) and about 304° C. (580° F.). Reaction temperature is operated low enough to prevent olefins in the FFA from polymerizing but high enough to foster olefin saturation, hydrodemetallation, hydrodeoxygenation, hydrodesulfurization and hydrodenitrification reactions to occur. Hydrodeoxygenation reactions preferably minimize hydrodecarbonylation and hydrodecarboxylation reactions to preserve carbon atoms on the paraffin chain.

The guard bed 27 can comprise a base metal catalyst on a support. Base metals useable in this process include non-noble metals, nickel, chromium, molybdenum and tungsten. Other base metals that can be used include tin, indium, germanium, lead, cobalt, gallium and zinc. The process can also use a metal sulfide, wherein the metal in the metal sulfide is selected from one or more of the base metals listed. The hydrotreating charge stream can be charged through the base metal catalysts at pressures from 1379 kPa (abs) (200 psia) to 13790 kPa (abs) (2000 psia). In a further embodiment, the guard bed catalyst can comprise a second metal, wherein the second metal includes one or more of the metals: tin, indium, ruthenium, rhodium, rhenium, osmium, iridium, germanium, lead, cobalt, gallium, zinc and thallium. A nickel molybdenum on alumina catalyst may be a suitable catalyst in the guard bed 27. Although only one guard bed is shown in FIG. 1, multiple guard beds may be contained in the hydrotreating reactor 28 such as 2, 3 or more and a hydrogen quench from a hydrogen manifold 18 may be injected at interbed locations to control temperature exotherms.

A contacted hydrocarbon stream is discharged from the guard bed 27. In the guard bed 27, most of the hydrodemetallation and hydrodeoxygenation reactions will occur with some hydrodenitrogenation and hydrodesulfurization occurring. Metals removed from biorenewable feedstocks will include alkali metals and alkali earth metals and phosphorous. If the guard bed has a dedicated reactor vessel, the contacted hydrocarbon stream will discharge from the guard bed reactor. However, in FIGURE, the guard bed 27 is contained in a hydrotreating reactor 28, so the contacted stream will receive a hydrogen quench from hydrogen manifold 18 and enter into a hydrotreating catalyst bed 29.

In the hydrotreating reactor 28, the contacted hydrocarbon stream is contacted with a hydrotreating catalyst in the hydrotreating catalyst bed 29 in the presence of hydrogen at hydrotreating conditions to saturate the olefinic or unsaturated portions of the n-paraffinic chains in the biorenewable feedstock. The hydrotreating catalyst also catalyzes hydrodeoxygenation reactions, including hydrodecarboxylation and hydrodecarbonylation reactions, to remove oxygenate functional groups from the hydrocarbon molecules in the biorenewable feedstock which are converted to water and carbon oxides. The hydrotreating catalyst also catalyzes hydrodesulfurization of organic sulfur and hydrodenitrogenation of organic nitrogen in the biorenewable feedstock. Essentially, the hydrotreating reaction removes heteroatoms from the hydrocarbons and saturates olefins in the feed stream.

The hydrotreating catalyst may be provided in one, two or more beds and employ interbed hydrogen quench streams from the hydrogen quench stream. Recycle hydrogen quench streams taken from the recycle hydrogen line 19 in a hydrogen manifold line 18 may be provided for interbed quench to the hydrotreating reactor 28. Three hydrotreating catalyst beds 29 are shown in FIG. 1, but one or more are contemplated.

The hydrotreating catalyst may comprise nickel, nickel/molybdenum, or cobalt/molybdenum dispersed on a high surface area support such as alumina. Other catalysts include one or more noble metals dispersed on a high surface area support. Non-limiting examples of noble metals include platinum and/or palladium dispersed on an alumina support such as gamma-alumina. Suitable hydrotreating catalysts include BDO 200 or BDO 300 or BDO 400 available from UOP LLC in Des Plaines, Illinois. The hydrotreating reaction temperature may range from between about 271° C. (520° F.) and about 427° C. (800° F.) and preferably between about 304° C. (580° F.) and about 400° C. (752° F.). Generally, hydrotreating conditions include a pressure of about 700 kPa (100 psig) to about 21 MPa (3000 psig).

A hydrotreated stream is produced in a hydrotreated line 32 from the hydrotreating reactor 28 comprising a hydrocarbon fraction which has a substantial n-paraffin concentration. Oxygenate concentration in the hydrocarbon fraction is essentially nil, whereas the olefin concentration is substantially reduced relative to the contacted biorenewable feed stream. The organic sulfur concentration in the hydrocarbon fraction is no more than 500 wppm and the organic nitrogen concentration in the hydrocarbon fraction is less than 10 wppm.

The hydrotreated stream in the hydrotreated line 32 may first flow to the combined isomerization feed exchanger 34 to heat the hydroisomerization feed stream in the hydroisomerization feed line 44 and cool the hydrotreated stream by heat exchange. As previously described, the cooled hydrotreated stream in the hydrotreated line 33 may then be heat exchanged with the hydrotreating charge hydrocarbon stream in line 26 in the combined feed heat exchanger 22 to further cool the cooled hydrotreated stream in the hydrotreated line 33 and heat the hydrotreating charge hydrocarbon stream in line 26. The twice cooled hydrotreated stream in line 35 may be even further cooled, perhaps to make steam, before it is separated.

The twice cooled hydrotreated stream in line 35 may be separated in a hydrotreating separator 36 which may comprise an enhanced hot separator (EHS) with the aid of a stripping gas fed in a stripping line 39 taken from an isomerization overhead line 58. The hydrotreated stream is separated to provide a hydrotreated vapor stream in a hydrotreated overhead line 38 and a hydrotreated liquid stream in a hydrotreated bottoms line 40 having a smaller oxygen concentration than the hydrotreating charge hydrocarbon stream in line 26. The hydrotreating separator 36 may be a high-pressure stripping column. In the hydrotreating separator 36, the hydrotreated stream from the hydrotreated line 32 flows down through the column where it is partially stripped of hydrogen, carbon dioxide, carbon monoxide, water vapor, propane, hydrogen sulfide, and phosphine, which are potential isomerization catalyst poisons, by contact with stripping gas from the stripping line 39. The stripping gas may comprise makeup hydrogen gas which has passed through the isomerization reactor 48 and an isomerization separator 56 as hereinafter described.

The stripping gas in the stripping line 39 enters the hydrotreating separator 36 below the inlet for the twice cooled hydrotreated stream in line 35. The hydrotreating separator 36 may include internals such as trays or packing located between the inlet for the hydrotreated stream in line 35 and the inlet for the stripping gas in the stripping line 39 to facilitate stripping of the hydrotreated stream. The stripping gas including stripped gases exits in a hydrotreated vapor stream in the hydrotreated overhead line 38 extending from a top of the hydrotreating separator 36 mixes with a cold aqueous stream in a cold aqueous line 63 from a boot of a cold separator 62 to provide a mixed stream in line 61. The mixed stream in line 61 is cooled in a cooler 64 and a cooled mixed stream in line 65 is combined with an isomerization liquid stream in an isomerization bottoms line 60 and a combined stream in line 66 enters the cold separator 62.

The hydrotreating separator 36 may operate at a temperature of about 177° C. (350° F.) to about 371° C. (700° F.) and preferably operates at a temperature of about 232° C. (450° F.) to about 315° C. (600° F.). The hydrotreating separator 36 may be operated at a slightly lower pressure than the hydrotreating reactor 28 accounting for pressure drop through intervening equipment. The hydrotreating separator 36 may be operated at pressures between about 3.4 MPa (gauge) (493 psig) and about 20.4 MPa (gauge) (2959 psig). The hydrotreated vapor stream in the hydrotreating separator overhead line 38 may have a temperature of the operating temperature of the hydrotreating separator 36.

The hydrotreated liquid stream which may have been stripped collects in the bottom of the hydrotreating separator 36 and flows in a hydrotreated bottoms line 40. The liquid hydrotreated stream comprises diesel range material, with a high paraffinic concentration if the hydrocarbon feed comprises a biorenewable feedstock. The liquid hydrotreated stream in the hydrotreating separator bottoms line 40 may be split into two streams: a hydroisomerization charge stream taken in a hydroisomerization charge line 42 and the recycle hydrotreated stream taken in the recycle line 16 both taken from the liquid hydrotreated stream in the hydrotreated bottoms line 40. The recycle hydrotreated stream in the recycle line 16 may be pumped and combined with the combined hydrocarbon stream in line 24 as previously described.

While a desired product, such as a transportation fuel, may be provided in the hydrotreated bottoms line 40 because the liquid hydrotreated stream comprises a higher concentration of normal paraffins. Accordingly, to improve the cold flow properties and reduce FBP, the hydrotreated liquid stream may be hydroisomerized.

Make-up hydrogen gas in make-up line 41 may be compressed in a make-up gas compressor 45 to provide compressed make up gas in a compressed make-up gas header 47. A hydroisomerization make-up gas stream is taken from the make-up gas header 47 in line 43 and mixed with the hydroisomerization charge stream in line 42 to provide a combined hydroisomerization charge stream in the combined hydroisomerization charge line 44. The combined hydroisomerization charge stream in the combined hydroisomerization charge line 44 may be heated in an hydroisomerization feed exchanger 34 by heat exchange with the hydrotreated stream in the hydrotreated line 32. A combined heated hydroisomerization charge stream in line 46 may be further heated in a hydroisomerization charge heater 37 to bring the combined hydroisomerization charge stream to hydroisomerization temperature before charging the combined hydroisomerization charge stream to the hydroisomerization reactor 49. A twice heated combined hydroisomerization charge stream in line 48 is passed to the hydroisomerization reactor 49.

Hydroisomerization, including hydrodewaxing, of the normal hydrocarbons in the hydroisomerization reactor 48 can be accomplished over one or more beds of hydroisomerization catalyst, and the hydroisomerization may be operated in a co-current mode of operation. Fixed bed, trickle bed down-flow or fixed bed liquid filled up-flow modes are both suitable.

The hydroisomerization catalyst comprises a dehydrogenation metal, a molecular sieve and a metal oxide binder. The hydroisomerization catalyst may comprise a dehydrogenation metal comprising a Group VIII metal. The dehydrogenation metal(s) may be selected from platinum, palladium, nickel, nickel molybdenum sulfide or nickel tungsten sulfide. Preferably, the dehydrogenation metal is selected from platinum or nickel tungsten sulfide. The concentration of dehydrogenation metal on the hydroisomerization catalyst may comprise from 0.05 to 5 wt % based on the transition metal(s).

The dehydrogenation metal is distributed between the molecular sieve and the binder with about 40 to about 65 wt %, preferably 45 to about 60 wt %, of the metals distributed on the molecular sieve and about 40 to about 65 wt %, preferably 45 to about 60 wt %, of the metals distributed on the binder. The associated benefit of the hydroisomerization catalyst is high activity and selectivity toward hydroisomerization. In a further embodiment the hydroisomerization catalyst further comprises less than about 0.5 wt % carbon with the associated benefit of high activity and selectivity towards hydroisomerization.

In an embodiment, the hydroisomerization catalyst comprises one or more molecular sieves having a topology selected from AEI, AEL, AFO, AFX, ATO, BEA, CHA, FAU, FER, MEL, MFI, MOR, MRE, MTT, MWW or TON, such as EU-2, ZSM-11, ZSM-22, ZSM-23, ZSM-48, SAPO-5, SAPO-11, SAPO-31, SAPO-34, SAPO-41, SSZ-13, SSZ-16, SSZ-39, MCM-22, zeolite Y, ferrierite, mordenite, ZSM-5 or zeolite beta, with the associated benefit of the molecular sieve being active in the hydroisomerization of linear hydrocarbons.

The metal oxide binder may be taken from the group comprising alumina, silica, silica-alumina and titania or mixtures thereof. Preferably the metal oxide binder is alumina and preferably it is gamma alumina.

The bydroisomerization catalyst may comprise a molecular sieve having an AEL topology and more specifically it may be SAPO-11. Most of the acid sites on SAPO-11 are weak to moderate acid sites. More specifically, at least 50% of the total acidity on the SAPO-11 is weak acidity and at least 60-80% of the external acidity on SAPO-11 is weak acidity.

The hydroisomerization catalyst typically comprises particles having a diameter of about 1 to about 5 millimeters. The catalyst production typically involves the formation of a stable, porous support, followed by impregnation of active metals. The stable, porous support typically comprises a metal oxide as well as a molecular sieve, which may be a zeolite. The stable support is produced with a high porosity, to ensure maximum surface area, and it is typically desired to disperse the active metal over the full internal and external surface area of the support. DI-200 available from UOP LLC in Des Plaines, Illinois may be a suitable hydroisomerization catalyst.

Hydroisomerization conditions generally include a temperature of about 150° C. (302° F.) to about 450° C. (842° F.) and a pressure of about 1724 kPa (abs) (250 psia) to about 13.8 MPa (abs) (2000 psia). In another embodiment, the hydroisomerization conditions include a temperature of about 300° C. (572° F.) to about 388° C. (730° F.), a pressure of about 3102 kPa (abs) (450 psia) to about 13790 kPa (abs) (2000 psia), a LHSV of about 0.5 to 3 hr⁻¹ and a hydrogen rate of about 337 Nm³/m³ (2,000 scf/bbl) to about 2,527 Nm³/m³ oil (15,000 scf/bbl).

A hydroisomerized stream in a hydroisomerized line 50 from the hydroisomerization reactor 49 is a branched-paraffin-rich stream. Preferably the hydroisomerized stream is predominantly a branched paraffin stream. It is envisioned that the hydroisomerized effluent may contain 80, 90 or 95 mass-% branched paraffins of the total paraffin content. Hydroisomerization conditions in the hydroisomerization reactor 49 are selected to avoid undesirable cracking, so the predominant product in the hydroisomerized stream in the hydroisomerized line 50 is a branched paraffin. By avoiding undesirable cracking, the hydroisomerized stream in the hydroisomerized line 50 will have near and only slightly less of the same composition with regard to carbon numbers as the hydroisomerization feed stream in the hydroisomerization charge line 42. The optimal amount of remaining normal paraffins in line 50 is dependent on the selectivity of the hydroisomerization catalysts but might typically be between 1-7 wt-%.

The hydroisomerized stream in the hydroisomerized line 50 from the hydroisomerization reactor 49 flows to an hydroisomerate exchanger 52 to be heat exchanged with a stripper liquid hydroisomerized stream in the cold bottoms line 70 to cool it before it is mixed with the vaporous hydrocracked stream in line 182 to provide a mixed hydroisomerized stream in line 54. The mixed hydroisomerized stream in line 54 is further cooled in a hydroisomerization cooler 55 and fed to the hydroisomerization separator 56 for separation into a liquid hydroisomerized stream and a vapor hydroisomerized stream. An internal packing may be located in the top of the hydroisomerization separator 56 to ensure liquid components are inhibited from leaving in a hydroisomerized overhead line 58. The vapor hydroisomerized stream in the hydroisomerized overhead line 58 extending from an overhead of hydroisomerization separator 56 may be compressed in the compressor 59 to provide the stripping gas in the stripping line 39 for the hydrotreating separator 36.

The liquid hydroisomerized stream in the hydroisomerization bottoms line 60 extending from a bottom of the hydroisomerization separator 56 may be fed to a stripping column 100 or to the product fractionation column 120 isolated from a hydrocracked liquid stream to be described hereinafter. In an embodiment, the liquid hydroisomerized stream in the hydroisomerization bottoms line 60 may be pumped to a cold separator 62 to be further separated along with a cooled vaporous hydrotreated stream in line 38 and a cold aqueous stream in the cold aqueous line 63 pumped around from the boot of the cold separator 62. The cold aqueous stream in line 63 may be combined with the vaporous hydrotreated stream in line 38 to provide a cooler hydrotreated stream in line 61. The cooler hydrotreated stream in line 61 may be cooled in a cooler 64. A further cooled hydrotreated stream in line 65 may be combined with the liquid hydroisomerized stream in line 60 to provide a cold separator feed stream in line 66 and fed to the cold separator 62.

In the cold separator 62, vaporous components in the hydroisomerized liquid stream and the hydrotreated vapor stream will separate and ascend to provide a cold vapor hydroisomerized stream in a cold overhead line 68 and a stripper liquid hydroisomerized stream in a cold bottoms line 70 and a cold aqueous stream taken in a cold aqueous line 63 from the boot. The cold vapor hydroisomerized steam in the cold overhead line 68 may be split between a recycle hydrogen stream in line 19 and a net cold vapor hydroisomerized stream in a net cold overhead line 72. The recycle hydrogen stream in line 19 is compressed in a recycle gas compressor 69 and recycled to the hydrotreating reactor 28 in the manifold line 18 for interbed quench and the hydrotreating hydrogen line 20 to the hydrocarbon stream in the feed line 12.

The net cold vapor hydroisomerized stream in the net cold overhead line 72 may be passed through a trayed or packed purge scrubbing column 74 where it is scrubbed by means of a scrubbing liquid such as an aqueous solution fed by scrubbing liquid line 76 to absorb acid gases including hydrogen sulfide and carbon dioxide by extracting them into the aqueous solution. Preferred scrubbing liquids include Selexol™ available from UOP LLC in Des Plaines, Illinois and amines such as alkanolamines including diethanol amine (DEA), monoethanol amine (MEA), methyl diethanol amine (MDEA), diisopropanol amine (DIPA), and diglycol amine (DGA). Other scrubbing liquids can be used in place of or in addition to the preferred amines. The lean scrubbing liquid contacts the cold vapor stream and absorbs acid gas contaminants. The resultant “sweetened” cold vapor stream is taken out from an overhead outlet of the purge scrubbing column 74 in a purge scrubber overhead line 78, and an acid gas rich scrubbing liquid is taken out from the bottoms at a bottom outlet of the purge scrubber column 74 in a purge scrubber bottoms line 80. The spent scrubbing liquid from the bottoms may be regenerated and recycled back to the purge scrubbing column 74 in the scrubbing liquid line 76. The scrubbed hydrogen-rich stream emerges from the purge scrubbing column 74 in the purge scrubber overhead line 78 and may be forwarded to a pressure-swing adsorption unit 82 or other hydrogen recovery plant to produce high purity hydrogen in line 84.

The purge scrubbing column 74 may be operated with a gas inlet temperature between about 38° C. (100° F.) and about 66° C. (150° F.) and an overhead pressure of about 3 MPa (gauge) (435 psig) to about 20 MPa (gauge) (2900 psig). Suitably, the purge scrubbing column 74 may be operated at a temperature of about 40° C. (104° F.) to about 125° C. (257° F.) and a pressure of about 1200 to about 1600 kPa. The temperature of the net cold vapor hydroisomerized stream 72 to the purge scrubbing column 74 may be between about 20° C. (68° F.) and about 80° C. (176° F.) and the temperature of the scrubbing liquid stream in the scrubbing liquid line 76 may be between about 20° C. (68° F.) and about 70° C. (158° F.).

Liquid hydroisomerized fuel components in the stripper liquid hydroisomerized stream from the liquid hydroisomerized stream and the vapor hydrotreated stream will exit the cold separator in the cold hydroisomerized bottoms line 70. The stripper liquid hydroisomerized stream in cold hydroisomerized bottoms line 70 comprises diesel and jet boiling range fuel components as well as other hydrocarbons such as propane, butane and naphtha.

In an embodiment, the stripper liquid hydroisomerized stream in the cold bottoms line 70 may be stripped in a cold stripping column 86 to remove hydrogen sulfide and other gases. The stripper liquid hydroisomerized stream in the cold bottoms line 70 may be heated by heat exchange in the hydroisomerate exchanger 52 with a hydroisomerized stream in the hydroisomerized line 50 to heat the cold liquid hydroisomerized stream and fed to the cold stripping column 86.

In an embodiment the cold stripping column 86 may be part of a dual-stripping vessel 100 that comprises a cold stripping column 86 and a hot stripping column 102 which are isolated from each other by an intervening wall 103. A stripping media which is an inert gas such as steam from a stripping media line 89 may be used to strip light gases from the stripper liquid hydroisomerized stream in line 70. The cold stripping column 86 provides an overhead stripping stream of naphtha, LPG, hydrogen, hydrogen sulfide, steam and other gases in a stripper overhead line 87 and a fractionator hydroisomerized stream in a cold stripped bottoms line 90. The overhead stripping stream in the overhead line 87 may be condensed by cooling and separated in a stripping receiver 95. A stripper overhead line 88 from the receiver 95 may carry a stripper overhead stream to an off-gas scrubber 140. Unstabilized liquid naphtha from the bottoms of the receiver 95 may be split to provide a reflux stream to the cold stripping column 86 and a stripper liquid overhead stream that may be transported in a stripper receiver bottoms line 96 to a debutanizer column 170 for naphtha and LPG recovery. A sour water stream may be collected from a boot of the overhead receiver 95.

The cold stripping column 86 may be operated with an overhead pressure of about 0.35 MPa (gauge) (50 psig), preferably no less than about 0.70 MPa (gauge) (100 psig), to no more than about 2.0 MPa (gauge) (290 psig). The temperature in the overhead receiver 95 ranges from about 38° C. (100° F.) to about 66° C. (150° F.) and the pressure is essentially the same as in the overhead of the cold stripping column 86. It is envisioned that the cold stripping column 86 and the hot stripping column 102 may be two completely separate vessels.

The fractionator liquid hydroisomerized stream in the cold stripper bottoms line 90 may be heated and fed to the product fractionation column 120. A fractionator liquid hydrocracked stream in a hot stripper bottoms line 106 may also be heated and fed to the product fractionation column 120.

A diesel stream in a bottoms line 124 is taken from a bottom of the product fractionation column 120. A hydrocracking charge stream in line 126 is taken from the diesel stream in the bottoms line 124 from the product fractionation column 120. The product fractionation column 120 may be reboiled by heat exchange with a suitable hot stream or in a fired heater 121 to provide the necessary heat for the distillation. Alternately, a stripping media which is an inert gas such as steam from a stripping media line may be used to heat the column. A reboil stream is taken to the fired heater 121 and returned boiling to the product fractionation column 120. A diesel product stream may be taken in a diesel product line 125 to a diesel pool and may be green diesel. The diesel stream in the distillation bottoms line 124 may be a diesel stream having a T5 of about 230° C. (446° F.) to about 296° C. (590° F.) and a T90 of about 343° C. (650° F.) to about 399° C. (750° F.).

The product fractionation column 120 provides an overhead gaseous stream of naphtha in an overhead line 122. The fractionation overhead stream may be completely condensed and separated from water in a fractionation receiver 130. Unstabilized liquid naphtha from the bottom of the receiver 130 in a fractionator overhead liquid line 132 may combined with a naphtha stream in line 176 while a condensed reflux stream is refluxed to the column. A sour water stream may be collected from a boot of the distillation receiver 130.

A kerosene stream may be taken from the side of the product fractionation column 120 in a side line 134. The kerosene stream taken in the side line 134 may be stripped in a kerosene stripper column 136 to drive off lower boiling materials which are returned back to the product fractionation column 120 at a higher elevation in an overhead kerosene line 135. A stripped bottoms kerosene stream is produced in a bottoms kerosene line 137, from which a boil up stream is reboiled and fed back to the kerosene stripper column 136 and a jet fuel product stream is taken in line 138. The jet fuel product stream in line 138 meets jet fuel specifications per ASTM D86 and may be a green jet fuel stream taken from a bottom of the kerosene stripper column 136. The jet fuel product stream in line 138 may be cooled and transported to the jet fuel pool.

The product fractionation column 120 may be operated with a bottoms temperature between about 149° C. (300° F.) and about 288° C. (550° F.), preferably no more than about 260° C. (500° F.), and an overhead pressure of about 0.35 MPa (gauge) (50 psig), preferably no less than about 0.70 MPa (gauge) (100 psig), to no more than about 2.0 MPa (gauge) (290 psig). The temperature in the overhead receiver 130 ranges from about 38° C. (100° F.) to about 66° C. (150° F.) and the pressure is essentially the same as in the overhead of the product fractionation column 120.

The overhead stripping stream of naphtha, LPG, hydrogen, hydrogen sulfide, steam and other gases in the stripper overhead line 88 may be passed through a trayed or packed off-gas scrubbing column 140 where it is scrubbed by means of a scrubbing liquid such as an aqueous solution fed by scrubbing liquid line 142 to remove acid gases including hydrogen sulfide and carbon dioxide by extracting them into the aqueous solution. Preferred scrubbing liquids include Selexol™ available from UOP LLC in Des Plaines, Illinois and amines such as alkanolamines including diethanol amine (DEA), monoethanol amine (MEA), methyl diethanol amine (MDEA), diisopropanol amine (DIPA), and diglycol amine (DGA). Other scrubbing liquids can be used in place of or in addition to the preferred amines. The lean scrubbing liquid contacts the overhead stripping stream and absorbs acid gas contaminants such as hydrogen sulfide and carbon dioxide. The resultant “sweetened” overhead stripping stream is taken out from an overhead outlet of the off-gas scrubbing column 140 in a recycle scrubber overhead line 144, and an acid gas rich scrubbing liquid is taken out from the bottoms at a bottom outlet of the recycle scrubber column 140 in a recycle scrubber bottoms line 146. The spent scrubbing liquid from the bottoms may be regenerated and recycled back to the off-gas scrubbing column 140 in the scrubbing liquid line 142. The scrubbed hydrocarbon-rich stream emerges from the off-gas scrubbing column 140 via the off-gas scrubber overhead line 144 and may be forwarded to the sponge absorber column 160 for hydrocarbon recovery.

The off-gas scrubbing column 140 may be operated with a gas inlet temperature between about 38° C. (100° F.) and about 66° C. (150° F.) and an overhead pressure of about 3 MPa (gauge) (435 psig) to about 20 MPa (gauge) (2900 psig). Suitably, the off-gas scrubbing column 140 may be operated at a temperature of about 40° C. (104° F.) to about 125° C. (257° F.) and a pressure of about 1200 to about 1600 kPa. The temperature of the overhead stripping stream 88 to the off-gas scrubbing column 140 may be between about 20° C. (68° F.) and about 80° C. (176° F.) and the temperature of the scrubbing liquid stream in the scrubbing liquid line 142 may be between about 20° C. (68° F.) and about 70° C. (158° F.).

The stripper liquid overhead stream in line 96 may be combined with a rich absorbent stream rich in LPG hydrocarbons in line 166 to provide a debutanizer column feed stream in line 97. The debutanizer column feed stream in line 97 is charged to the debutanizer column 170. In an embodiment, the debutanizer column 170 may fractionate the stripper liquid overhead stream and the rich absorbent stream into a debutanized bottoms stream comprising predominantly C₅₊ hydrocarbons and a debutanizer overhead stream comprising LPG hydrocarbons including C3-C4 hydrocarbons. The debutanizer overhead stream in a debutanizer overhead line 172 may be fully condensed with reflux to the debutanizer column 170 and recovery of LPG in a debutanized overhead liquid stream in a debutanizer net receiver bottoms line 174. The debutanized bottoms stream may be withdrawn from a bottom of the debutanizer column 170 in a debutanized bottoms line 176. A reboil stream taken from a debutanized bottoms stream in a debutanizer bottoms line 175 from a bottom of the debutanizer column 170 may be boiled up in the reboil line and sent back to the debutanizer column 170 to provide heat to the column. Alternatively, a hot inert media stream such as steam may be fed to the column 170 to provide heat. A net debutanized bottoms stream in line 176 comprising naphtha may be split between a lean absorbent stream in the lean absorbent line 162 and a product naphtha stream which is cooled and forwarded to a gasoline pool in line 178. In an exemplary embodiment, the debutanizer column 170 may be operated at an overhead pressure of from about 0 kPa (a) (0 psia) to about 2758 kPa (a) (400 psia).

Hydrogen sulfide and C3 hydrocarbons both co-boil and their separation is considered to be difficult. A typical process of producing the liquefied petroleum gas requires an amine treatment of the liquefied petroleum gas. Amine treatment is employed to meet the hydrogen sulfide specification of the produced liquefied petroleum gas. A permissible level of hydrogen sulfide may be less than 30 wppm. For amine treatment, an amine regenerator is also required. Typically, the debutanized overhead liquid stream in the net receiver bottoms line 174 would be treated in an amine treating unit to separate hydrogen sulfide and produce a liquefied petroleum gas product stream. The present process comprises a deethanizer column 190 to separate hydrogen sulfide from the debutanized overhead liquid stream. The hydrogen sulfide is separated in the overhead stream of the deethanizer column 190 which is recycled to the sponge absorber column 160. The liquefied petroleum gas product stream is taken from the bottoms of the deethanizer column 190. The present process of producing liquefied petroleum gas is an economical process over a conventional process that uses an amine treating unit to purify the liquefied petroleum gas stream requiring about 23% less capital expenditure.

In accordance with the present disclosure, the debutanized overhead liquid stream in the net receiver bottoms line 174 is pumped to the deethanizer column 190. The debutanized overhead liquid stream is fractionated in the deethanizer column 190 to separate light gases including a bulk of the hydrogen sulfide from the C3-C4 hydrocarbons. The deethanizer column 190 is operated at an overhead pressure to recover the C3-C4 hydrocarbons and separate a majority of the hydrogen sulfide in the deethanizer column overhead stream. In an exemplary embodiment, the deethanizer column 190 is operated at an overhead pressure of about 2068 kPa (gauge) (300 psig) to about 3450 kPa (gauge) (500 psig). In another exemplary embodiment, the deethanizer column 190 is operated at an overhead pressure of about 2413 kPa (gauge) (350 psig) to about 3102 kPa (gauge) (450 psig). In an aspect, the deethanizer column 190 is operated at a higher overhead pressure than the debutanizer column 170.

A deethanizer column overhead stream is taken in line 191 of the deethanizer column 190 and passed to an overhead receiver 189. A net deethanizer column overhead stream is taken in line 192. The net gaseous deethanizer column overhead stream in line 192 comprises C2-hydrocarbons and some C3 hydrocarbons may be present in this stream. C3 hydrocarbons co-boil with hydrogen sulfide, so the C3 hydrocarbons tend to go up with the hydrogen sulfide in the overhead stream in line 191. The C3 hydrocarbons should be separated from the net deethanizer column overhead stream in line 192 to increase liquefied petroleum gas recovery. In an embodiment, the net deethanizer column overhead stream in line 192 is charged to the sponge absorber column 160 to recover valuable C3 hydrocarbons that would otherwise be lost with the hydrogen sulfide in the deethanizer overhead.

In an exemplary embodiment, the net deethanizer column overhead stream in line 192 may be combined with the scrubbed hydrocarbon-rich stream in the off-gas scrubber overhead line 144 to provide a combined off-gas stream in line 145. The combined off-gas stream in line 145 is charged to the sponge absorber column 160. A lean absorbent stream in a lean absorbent line 162 may be fed into the sponge absorber column 160 through an absorbent inlet. The lean absorbent may comprise a naphtha stream in a lean absorbent line 162 perhaps from the debutanizer bottoms stream in line 176. In the sponge absorber column 160, the lean absorbent stream is counter-currently contacted with the net deethanizer column overhead stream in line 192 and the scrubbed hydrocarbon-rich stream in line 144. The sponge absorbent absorbs LPG hydrocarbons from the net deethanizer column overhead stream in line 192 and the scrubbed hydrocarbon-rich stream in line 144 into an absorbent rich stream.

The hydrocarbons absorbed by the sponge absorbent include some methane and ethane and most of the LPG, C₃ and C₄, hydrocarbons, and any C₅ and C₆₊ light naphtha hydrocarbons in the net stripper gaseous stream. The sponge absorber column 160 operates at a temperature of about 34° C. (93° F.) to about 60° C. (140° F.) and a pressure essentially the same as or lower than the off-gas scrubbing column 140 less frictional losses. A sponge absorption off gas stream depleted of LPG hydrocarbons is withdrawn from a top of the sponge absorber column 160 at an overhead outlet through a sponge absorber overhead line 164. The sponge absorption off gas stream in the sponge absorber overhead line 164 may be transported to a fuel gas header that is not shown for providing fuel gas needs. A rich absorbent stream rich in LPG hydrocarbons is withdrawn in a rich absorber bottoms line 166 from a bottom of the sponge absorber column 160 at a bottoms outlet which may be fed to a debutanizer column 170 via the stripper overhead liquid stream in the stripper receiver bottoms line 96 as previously described.

Referring back to the deethanizer column 190, a deethanized stream comprising C3-C4 hydrocarbons is withdrawn from the bottoms of the deethanizer column 190. A reboil stream is taken in line 195 and heated in a reboiler 91 with a low-pressure steam stream in line 198. The low-pressure steam is less expensive and readily available to provide heat to the bottoms of the deethanizer column 190 for fractionation.

A liquefied petroleum gas product stream may be taken in line 197 from the deethanized bottoms stream in line 194. In an aspect, the liquefied petroleum gas product stream in line 197 comprises C3-C4 hydrocarbons and about 1 to about 30 wppm hydrogen sulfide. In another aspect, the liquefied petroleum gas product stream in line 197 comprises C3-C4 hydrocarbons and less than about 20 wppm hydrogen sulfide. In yet another aspect, the liquefied petroleum gas product stream in line 197 comprises C3-C4 hydrocarbons and less than about 5 wppm hydrogen sulfide. A portion or the entirety of the liquefied petroleum gas product stream in line 197 may be charged to a hydrogen generation unit. A portion or the entirety of the liquefied petroleum gas product stream in line 197 may be charged to a storage tank and/or made available for purchase.

The hydrocracking charge stream in the hydrocracking charge line 126 may be reserved in a hydrocracking charge surge drum 127 and pumped in line 128 to the hydrocracking reactor 150. The hydrocracking reactor 150 is downstream of the hydroisomerization reactor 48. The hydrocracking charge stream in line 128 may be mixed with a hydrocracking hydrogen stream in line 152 taken from the compressed make-up hydrogen stream in compressed make-up gas header 47 to provide combined hydrocracking charge stream in a combined hydrocracking charge line 154, heated by heat exchanged with a hydrocracked stream in line 158 in a hydrocracking effluent feed heater 155 and charged in line 156 to the hydrocracking reactor 150.

The hydrocracking reactor 150 may be a fixed bed reactor that comprises one or more vessels, single or multiple catalyst beds in each vessel, and various combinations of hydrocracking catalyst in one or more vessels. The hydrocracking reactor 150 may be operated in a conventional continuous gas phase, a moving bed or a fluidized bed hydroprocessing reactor.

The combined hydrocracking stream in line 156 is hydrocracked over a hydrocracking catalyst in the hydrocracking reactor 150 in the presence of a hydrocracking hydrogen stream from a hydrocracking hydrogen line 152 to provide a hydrocracked stream. A portion of the hydrocracking charge stream in line 156 may be used as an interbed quench to cool hydrocracked effluent between catalyst beds. In an alternative aspect, the recycle hydrogen from the recycle hydrogen line 19 may be added between hydrocracking catalyst beds.

The hydrocracking reactor may provide a total conversion of at least about 20 vol % and typically greater than about 60 vol % of the hydrocracking charge stream in the hydrocracking charge line 126 to products boiling below the heavy diesel range of about 293° C. (560° F.) to about 310° C. (590° F.). The hydrocracking reactor 150 may operate at partial conversion of more than about 30 vol % or full conversion of at least about 90 vol % of the feed based on total conversion. The hydrocracking reactor 150 may be operated at mild hydrocracking conditions which will provide about 20 to about 60 vol %, preferably about 20 to about 50 vol %, total conversion of the hydrocracking charge stream to product boiling below the heavy diesel boiling range.

The hydrocracking catalyst may utilize amorphous silica-alumina bases or zeolite bases combined with one or more Group VIII or Group VIB metal hydrogenating components to selectively produce a balance of light diesel and jet fuel distillate. In another aspect, a catalyst which comprises, in general, any crystalline zeolite cracking base upon which is deposited a Group VIII metal hydrogenating component may be suitable. Additional hydrogenating components may be selected from Group VIB for incorporation with the zeolite base. Moreover, the hydroisomerization catalyst from the hydroisomerization reactor 48 can be used as hydrocracking catalyst in the hydrocracking reactor 150 but run at the high end of the hydroisomerization temperature range.

The zeolite cracking bases are sometimes referred to in the art as molecular sieves and are usually composed of silica, alumina and one or more exchangeable cations such as sodium, magnesium, calcium, rare earth metals, etc. They are further characterized by crystal pores of relatively uniform diameter between about 4 and about 14 Angstroms. It is preferred to employ zeolites having a relatively high silica/alumina mole ratio between about 3 and about 12. Suitable zeolites found in nature include, for example, mordenite, stilbite, heulandite, ferrierite, dachiardite, chabazite, erionite and faujasite. Suitable synthetic zeolites include, for example, the B, X, Y and L crystal types, e.g., synthetic faujasite and mordenite. The preferred zeolites are those having crystal pore diameters between about 8 and 12 Angstroms, wherein the silica/alumina mole ratio is about 4 to 6. One example of a zeolite falling in the preferred group is synthetic Y molecular sieve.

The natural occurring zeolites are normally found in a sodium form, an alkaline earth metal form, or mixed forms. The synthetic zeolites are nearly always prepared in the sodium form. In any case, for use as a cracking base it is preferred that most or all of the original zeolitic monovalent metals be ion-exchanged with a polyvalent metal and/or with an ammonium salt followed by heating to decompose the ammonium ions associated with the zeolite, leaving in their place hydrogen ions and/or exchange sites which have actually been decationized by further removal of water. Hydrogen or “decationized” Y zeolites of this nature are more particularly described in U.S. Pat. No. 3,100,006.

Mixed polyvalent metal-hydrogen zeolites may be prepared by ion-exchanging with an ammonium salt, then partially back exchanging with a polyvalent metal salt and then calcining. In some cases, as in the case of synthetic mordenite, the hydrogen forms can be prepared by direct acid treatment of the alkali metal zeolites. In one aspect, the preferred cracking bases are those which are at least about 10 wt %, and preferably at least about 20 wt %, metal-cation-deficient, based on the initial ion-exchange capacity. In another aspect, a desirable and stable class of zeolites is one wherein at least about 20 wt % of the ion exchange capacity is satisfied by hydrogen ions.

The active metals employed in the preferred hydrocracking catalysts of the present disclosure as hydrogenation components are those of Group VIII, i.e., iron, cobalt, nickel, ruthenium, rhodium, palladium, osmium, iridium and platinum. In addition to these metals, other promoters may also be employed in conjunction therewith, including the metals of Group VIB, e.g., molybdenum and tungsten. The amount of hydrogenating metal in the catalyst can vary within wide ranges. Broadly speaking, any amount between about 0.05 wt % and about 30 wt % may be used. In the case of the noble metals, it is normally preferred to use about 0.05 to about 2 wt % noble metal. Noble metals may be preferred as the hydrogenation metal on the hydrocracking catalyst to provide selectivity to jet fuel due to the absence of hydrogen sulfide and ammonia which can deactivate noble metal catalysts, but which have been removed upstream in the process.

The method for incorporating the hydrogenation metal is to contact the base material with an aqueous solution of a suitable compound of the desired metal wherein the metal is present in a cationic form. Following addition of the selected hydrogenation metal or metals, the resulting catalyst powder is then filtered, dried, pelleted with added lubricants, binders or the like if desired, and calcined in air at temperatures of, e.g., about 371° C. (700° F.) to about 648° C. (200° F.) in order to activate the catalyst and decompose ammonium ions. Alternatively, the base component may be pelleted, followed by the addition of the hydrogenation component and activation by calcining.

The foregoing catalysts may be employed in undiluted form, or the powdered catalyst may be mixed and copelleted with other relatively less active catalysts, diluents or binders such as alumina, silica gel, silica-alumina cogels, activated clays and the like in proportions ranging between about 5 and about 90 wt %. These diluents may be employed as such, or they may contain a minor proportion of an added hydrogenating metal such as a Group VIB and/or Group VIII metal. Additional metal promoted hydrocracking catalysts may also be utilized in the process of the present disclosure which comprises, for example, aluminophosphate molecular sieves, crystalline chromosilicates and other crystalline silicates. Crystalline chromosilicates are more fully described in U.S. Pat. No. 4,363,178.

By one approach, the hydrocracking conditions may include a temperature from about 290° C. (550° F.) to about 468° C. (875° F.), preferably 300° C. (572° F.) to about 445° C. (833° F.), a pressure from about 2.7 MPa (gauge) (400 psig) to about 20.7 MPa (gauge) (3000 psig), a liquid hourly space velocity (LHSV) from about 0.4 to less than about 2.5 hr⁻¹ and a hydrogen rate of about 337 Nm³/m³ (2,000 scf/bbl) to about 2,527 Nm³/m³ oil (15,000 scf/bbl).

The hydrocracked stream may exit the hydrocracking reactor 150 in a hydrocracked line 158. In an embodiment, the hydrocracked stream may be cooled by heat exchange with the combined hydrocracking charge stream in line 154 and transported to a hydrocracking separator 180 to be separated. The hydrocracked stream may be separated in a hydrocracking separator 180 to provide a vapor hydrocracked stream in a hydrocracking separator overhead line 182 and a liquid hydrocracked stream comprising a stripper liquid hydrocracked stream in a hydrocracking separator bottoms line 184. The hydrocracking separator 180 may be in downstream communication with the hydrocracking reactor 150.

The hydrocracking separator 180 operates at about 177° C. (350° F.) to about 371° C. (700° F.) and preferably operates at about 232° C. (450° F.) to about 315° C. (600° F.). The hydrocracking separator 180 may be operated at a slightly lower pressure than the hydrocracking reactor 150 accounting for pressure drop through intervening equipment. The hydrocracking separator 180 may be operated at pressures between about 3.4 MPa (gauge) (493 psig) and about 20.4 MPa (gauge) (2959 psig). The vapor hydrocracked stream in the hydrocracking separator overhead line 182 may have a temperature of the operating temperature of the hydrocracking separator 180. The vaporous hydrocracked stream in the hydrocracking separator overhead line 182 may be combined with the cooled hydroisomerized stream in the hydroisomerized line 50 to provide a mixed hydroisomerized stream in line 54 and separated together in the hydroisomerization separator 56.

The stripper liquid hydrocracked stream in line 184 may be fed to the hot stripping column 102 to be stripped of lighter gases. In an embodiment, the stripper liquid hydrocracked stream in line 184 may be stripped in the hot stripping column 102 to remove hydrogen sulfide, naphtha and lighter gases. The stripper liquid hydrocracked stream in the hydrocracked bottoms line 184 may be fed directly to the hot stripping column 102 without undergoing cooling. The hot stripping column 102 may be operated with a bottoms temperature between about 149° C. (300° F.) and about 288° C. (550° F.), preferably no more than about 260° C. (500° F.).

In an embodiment, the hot stripping column 102 may be part of a dual-stripping vessel 100 that comprises the cold stripping column 86 and the hot stripping column 102 which are isolated from each other by an intervening wall 103. A stripping media which is an inert gas such as steam from a hot stripping media line 104 may be used to strip light gases from the stripper liquid hydrocracked stream in line 184. The hot stripping column 102 provides an overhead stripping stream of naphtha, LPG, hydrogen, hydrogen sulfide, steam and other gases in a hot stripper overhead line 105 and a fractionator liquid hydrocracked stream in a hot stripped bottoms line 106. The hot stripper overhead line 105 is preferably fed to the cold stripping column 86 to be further stripped of lighter boiling components. The fractionator liquid hydrocracked stream in the hot stripped line 106 is heated preferably without a fired heater and fed to the product fractionation column 120 at a feed location that is above the feed location for the fractionator liquid hydroisomerized stream in cold stripped bottoms line 90. The fractionator liquid hydrocracked stream is fractionated in the product fractionation column 120 with the fractionator liquid hydroisomerized stream as described previously.

### Example

Two different feedstocks were processed for producing liquefied petroleum gas. The first feed was 100 wt % soybean oil and the second feed was 70 wt % soybean oil and 30 wt % animal fat. The feeds were processed for two different modes of operation. The 100 wt % soybean oil was processed for diesel production mode. The 70 wt % soybean oil and 30 wt % animal fat feed was processed for diesel production mode and for jet fuel production mode. For comparative study, the present process with the deethanizer column was compared with another process with the amine treater for producing liquefied petroleum gas for different modes of operation. Results of the study are summarized in table below.

TABLE

| | | H2S in LPG | H2S in LPG by |
|---|---|---|---|
| | | by amine | deethanizer |
| | Mode of | treatment, | column, |
| Feed, wt % | operation | wppm | wppm |
| 100% soyabean oil | diesel production | 25 | 6 |
| | mode | | |
| 70% soybean oil | diesel production | 24 | 4 |
| and 30% animal fat | mode | | |
| 100% soybean oil | jet fuel production | 16 | 4 |
| | mode | | |

As evident from the table, the liquefied petroleum gas processed by the deethanizer column had a much lower hydrogen sulfide concentration than the liquefied petroleum gas processed by the amine treater. Processing biorenewable feed, the liquefied petroleum gas processed with the deethanizer column under various modes of operation had hydrogen sulfide concentration below 10 wppm.

### Specific Embodiments

While the following is described in conjunction with specific embodiments, it will be understood that this description is intended to illustrate and not limit the scope of the preceding description and the appended claims.

A first embodiment of the present disclosure is a process of producing liquefied petroleum gas, the process comprising fractionating a stream comprising C4-hydrocarbons and at least 10 wppm hydrogen sulfide in a fractionation column to provide an overhead stream comprising C2-hydrocarbons and a majority of the hydrogen sulfide and a bottoms stream comprising C3-C4 hydrocarbons and about 1 to about 30 wppm hydrogen sulfide. An embodiment of the present disclosure is one, any or all of prior embodiments in this paragraph up through the first embodiment in this paragraph further comprising fractionating a hydrocarbon stream comprising naphtha range hydrocarbons in a first fractionation column to provide the stream comprising C4-hydrocarbon. An embodiment of the present disclosure is one, any or all of prior embodiments in this paragraph up through the first embodiment in this paragraph further comprising passing the overhead stream to a sponge absorber to provide a contacted bottoms stream comprising C3-hydrocarbons. An embodiment of the present disclosure is one, any or all of prior embodiments in this paragraph up through the first embodiment in this paragraph, wherein the hydrocarbon stream is taken from a hydroisomerized stream. An embodiment of the present disclosure is one, any or all of prior embodiments in this paragraph up through the first embodiment in this paragraph, wherein the fractionation column is operated at a pressure of about 2068 kPa to about 3450 kPa. An embodiment of the present disclosure is one, any or all of prior embodiments in this paragraph up through the first embodiment in this paragraph further comprising contacting the overhead stream with an absorbent stream in the sponge absorber to provide the contacted bottoms stream comprising C2-hydrocarbons; and charging the contacted bottoms stream to the fractionation column. An embodiment of the present disclosure is one, any or all of prior embodiments in this paragraph up through the first embodiment in this paragraph, wherein the bottoms stream comprises less than 5 wppm hydrogen sulfide. An embodiment of the present disclosure is one, any or all of prior embodiments in this paragraph up through the first embodiment in this paragraph further comprising passing a low-pressure steam to a reboiler of the fractionation column to provide heat for fractionating the stream. An embodiment of the present disclosure is one, any or all of prior embodiments in this paragraph up through the first embodiment in this paragraph further comprising stripping the hydroisomerized stream in a stripping column to produce a stripped stream and a stripping column overhead stream comprising naphtha range hydrocarbons; and taking the hydrocarbon stream from the stripping column overhead stream. An embodiment of the present disclosure is one, any or all of prior embodiments in this paragraph up through the first embodiment in this paragraph further comprising separating the striping column overhead stream into an overhead vapor stream and an overhead liquid stream; and fractionating the overhead liquid stream in the first fractionation column to provide the stream comprising C4-hydrocarbons. An embodiment of the present disclosure is one, any or all of prior embodiments in this paragraph up through the first embodiment in this paragraph, wherein the hydroisomerized stream is produced from a biorenewable feedstock.

A second embodiment of the present disclosure is a process for producing liquefied petroleum gas, the process comprising fractionating a hydroisomerized stream comprising naphtha range hydrocarbons in a first fractionation column to provide a first overhead stream comprising C4-hydrocarbons and hydrogen sulfide; and fractionating the first overhead stream in a second fractionation column to provide a second overhead stream comprising C2-hydrocarbons and a majority of the hydrogen sulfide and a second bottoms stream comprising C3-C4 hydrocarbons and about 1 to about 30 wppm hydrogen sulfide. An embodiment of the present disclosure is one, any or all of prior embodiments in this paragraph up through the second embodiment in this paragraph, wherein the second bottoms stream comprises less than 5 wppm hydrogen sulfide. An embodiment of the present disclosure is one, any or all of prior embodiments in this paragraph up through the second embodiment in this paragraph, wherein the second fractionation column is operated at a lower pressure than the first fractionation column. An embodiment of the present disclosure is one, any or all of prior embodiments in this paragraph up through the second embodiment in this paragraph, wherein the second fractionation column is operated at a pressure of about 2068 kPa to about 3450 kPa. An embodiment of the present disclosure is one, any or all of prior embodiments in this paragraph up through the second embodiment in this paragraph further comprising contacting the second overhead stream with an absorbent stream in the sponge absorber to provide a contacted bottoms stream comprising C2-hydrocarbons; and charging the contacted bottoms stream to the first fractionation column. An embodiment of the present disclosure is one, any or all of prior embodiments in this paragraph up through the second embodiment in this paragraph, wherein the hydroisomerized stream is produced from a biorenewable feedstock.

A third embodiment of the present disclosure is a process of producing liquefied petroleum gas, the process comprising fractionating a C4-hydrocarbon stream comprising at least 10 wppm hydrogen sulfide in a fractionation column at a pressure of about 2068 kPa to about 3450 kPa to provide an overhead stream comprising C2-hydrocarbons and a bottoms stream comprising C3-C4 hydrocarbons and about 1 to about 30 wppm hydrogen sulfide; and passing the second overhead stream to a sponge absorber to provide a contacted bottoms stream comprising C2-hydrocarbons. An embodiment of the present disclosure is one, any or all of prior embodiments in this paragraph up through the third embodiment in this paragraph, wherein the bottoms stream comprises less than 5 wppm hydrogen sulfide. An embodiment of the present disclosure is one, any or all of prior embodiments in this paragraph up through the third embodiment in this paragraph further comprising fractionating a hydrocarbon stream comprising naphtha range hydrocarbons in a first fractionation column to provide the stream comprising C4-hydrocarbon.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process of producing liquefied petroleum gas, the process comprising:
fractionating a stream comprising C4-hydrocarbons and at least 10 wppm hydrogen sulfide in a fractionation column to provide an overhead stream comprising C2-hydrocarbons and a majority of the hydrogen sulfide and a bottoms stream comprising C3-C4 hydrocarbons and about 1 to about 30 wppm hydrogen sulfide.

2. The process of claim 1 further comprising fractionating a hydrocarbon stream comprising naphtha range hydrocarbons in a first fractionation column to provide said stream comprising C4-hydrocarbon.

3. The process of claim 1 further comprising passing said overhead stream to a sponge absorber to provide a contacted bottoms stream comprising C3-hydrocarbons.

4. The process of claim 2, wherein said hydrocarbon stream is taken from a hydroisomerized stream.

5. The process of claim 1, wherein the fractionation column is operated at a pressure of about 2068 kPa to about 3450 kPa.

6. The process of claim 3 further comprising:
contacting said overhead stream with an absorbent stream in the sponge absorber to provide said contacted bottoms stream comprising C2-hydrocarbons; and
charging said contacted bottoms stream to the fractionation column.

7. The process of claim 1, wherein said bottoms stream comprises less than 5 wppm hydrogen sulfide.

8. The process of claim 1 further comprising passing a low-pressure steam to a reboiler of the fractionation column to provide heat for fractionating said stream.

9. The process of claim 4 further comprising:
stripping said hydroisomerized stream in a stripping column to produce a stripped stream and a stripping column overhead stream comprising naphtha range hydrocarbons; and
taking said hydrocarbon stream from said stripping column overhead stream.

10. The process of claim 9 further comprising:
separating said striping column overhead stream into an overhead vapor stream and an overhead liquid stream; and
fractionating said overhead liquid stream in the first fractionation column to provide said stream comprising C4-hydrocarbons.

11. The process of claim 4, wherein said hydroisomerized stream is produced from a biorenewable feedstock.

12. A process for producing liquefied petroleum gas, the process comprising:
fractionating a hydroisomerized stream comprising naphtha range hydrocarbons in a first fractionation column to provide a first overhead stream comprising C4-hydrocarbons and hydrogen sulfide; and
fractionating said first overhead stream in a second fractionation column to provide a second overhead stream comprising C2-hydrocarbons and a majority of the hydrogen sulfide and a second bottoms stream comprising C3-C4 hydrocarbons and about 1 to about 30 wppm hydrogen sulfide.

13. The process of claim 12, wherein said second bottoms stream comprises less than 5 wppm hydrogen sulfide.

14. The process of claim 12, wherein the second fractionation column is operated at a lower pressure than the first fractionation column.

15. The process of claim 12, wherein the second fractionation column is operated at a pressure of about 2068 kPa to about 3450 kPa.

16. The process of claim 12 further comprising:
contacting said second overhead stream with an absorbent stream in the sponge absorber to provide a contacted bottoms stream comprising C2-hydrocarbons; and
charging said contacted bottoms stream to the first fractionation column.

17. The process of claim 12, wherein said hydroisomerized stream is produced from a biorenewable feedstock.

18. A process of producing liquefied petroleum gas, the process comprising:
fractionating a C4-hydrocarbon stream comprising at least 10 wppm hydrogen sulfide in a fractionation column at a pressure of about 2068 kPa to about 3450 kPa to provide an overhead stream comprising C2-hydrocarbons and a bottoms stream comprising C3-C4 hydrocarbons and about 1 to about 30 wppm hydrogen sulfide; and
passing said second overhead stream to a sponge absorber to provide a contacted bottoms stream comprising C2-hydrocarbons.

19. The process of claim 18, wherein said bottoms stream comprises less than 5 wppm hydrogen sulfide.

20. The process of claim 18 further comprising fractionating a hydrocarbon stream comprising naphtha range hydrocarbons in a first fractionation column to provide said stream comprising C4-hydrocarbon.
